(19) United States
(12) Patent Application Publication
(10) Pub. No.: US 2018/0016609 A1
(43) Pub. Date: Jan. 18, 2018

(54) A Process for the Preparation of Nucleic Acid by Means of 3'-O-Azidomethyl Nucleotide Triphosphate

(71) Applicant: Nuclera Nucleics Ltd., Cambridge (GB)

(72) Inventors: Michael C. Chen, Cambridge (GB); Radu A. Lazar, Cambridge (GB); Jiahao Huang, Cambridge (GB); Gordon R. McInroy, Cambridge (GB)

(21) Appl. No.: 15/555,232

(22) PCT Filed: Mar. 3, 2016
(86) PCT No.: PCT/GB2016/050555

§ 371 Date: Sep. 1, 2017

(30) Foreign Application Priority Data
Mar. 3, 2015 (GB) 1503534.8

(51) Int. Cl. C12P 19/34; C07H 19/12; C07H 19/10; C07H 19/20; C07H 1/00

(52) U.S. Cl. CPC C12P 19/34; C12Y 207/07031; C07H 19/20; C07H 1/00; C07H 19/10; C07H 19/12

(57) ABSTRACT

The invention relates to a method of nucleic acid synthesis comprising the use of 3′-O-azidomethyl blocked nucleotide triphosphates which comprises the step of adding a capping group to any uncleaved 3′-O-azidomethyl groups and to the use of kits comprising said capping groups in a method of nucleic acid synthesis. The invention also relates to capped nucleotide triphosphates and 3′-O-azidomethyl capping groups.

### FIELD OF THE INVENTION

The invention relates to a method of nucleic acid synthesis comprising the use of 3′-O-azidomethyl blocked nucleotide triphosphates which comprises the step of adding a capping group to any uncleaved 3′-O-azidomethyl groups and to the use of kits comprising said capping groups in a method of nucleic acid synthesis. The invention also relates to capped nucleotide triphosphates and 3′-O-azidomethyl capping groups.

### BACKGROUND OF THE INVENTION

Nucleic acid synthesis is vital to modern biotechnology. The rapid pace of development in the biotechnology arena has been made possible by the scientific community's ability to artificially synthesise DNA, RNA and proteins.

Artificial DNA synthesis—a £1 billion and growing market—allows biotechnology and pharmaceutical companies to develop a range of peptide therapeutics, such as insulin for the treatment of diabetes. It allows researchers to characterise cellular proteins to develop new small molecule therapies for the treatment of diseases our aging population faces today, such as heart disease and cancer. It even paves the way forward to creating life, as the Venter Institute demonstrated in 2010 when they placed an artificially synthesised genome into a bacterial cell.

However, current DNA synthesis technology does not meet the demands of the biotechnology industry. While the benefits of DNA synthesis are numerous, an oft-mentioned problem prevents the further growth of the artificial DNA synthesis industry, and thus the biotechnology field. Despite being a mature technology, it is practically impossible to synthesise a DNA strand greater than 200 nucleotides in length, and most DNA synthesis companies only offer up to 120 nucleotides. In comparison, an average protein-coding gene is of the order of 2000-3000 nucleotides, and an average eukaryotic genome numbers in the billions of nucleotides. Thus, all major gene synthesis companies today rely on variations of a ‘synthesise and stitch’ technique, where overlapping 40-60-mer fragments are synthesised and stitched together by PCR (see Young, L. et al. (2004) Nucleic Acid Res. 32, e59). Current methods offered by the gene synthesis industry generally allow up to 3 kb in length for routine production.

The reason DNA cannot be synthesised beyond 120-200 nucleotides at a time is due to the current methodology for generating DNA, which uses synthetic chemistry (i.e., phosphoramidite technology) to couple a nucleotide one at a time to make DNA. As the efficiency of each nucleotide-coupling step is 95.0-99.0% efficient, it is mathematically impossible to synthesise DNA longer than 200 nucleotides in acceptable yields. The Venter Institute illustrated this laborious process by spending 4 years and 20 million USD to synthesise the relatively small genome of a bacterium (see Gibson, D. G. et al. (2010) Science 329, 52-56).

Known methods of DNA sequencing use template-dependent DNA polymerases to add 3′-reversibly terminated nucleotides to a growing double-stranded substrate (see, Bentley, D. R. et al. (2008) Nature 456, 53-59). In the ‘sequencing-by-synthesis’ process, each added nucleotide contains a dye, allowing the user to identify the exact sequence of the template strand. Albeit on double-stranded DNA, this technology is able to produce strands of between 500-1000 bps long. However, this technology is not suitable for de novo nucleic acid synthesis because of the requirement for an existing nucleic acid strand to act as a template.

There is therefore a need to provide an improved method of nucleic acid synthesis that is able to overcome the problems associated with currently available methods.

### SUMMARY OF THE INVENTION

According to a first aspect of the invention, there is provided a method of nucleic acid synthesis, which comprises the steps of:
(a) providing an initiator sequence;
(b) adding a 3′-O-azidomethyl blocked nucleotide triphosphate to said initiator sequence in the presence of terminal deoxynucleotidyl transferase (TdT) or a functional equivalent or fragment thereof,
(c) removal of TdT;
(d) cleaving the 3′-O-azidomethyl group from the 3′-O-azidomethyl blocked nucleotide triphosphate in the presence of a cleaving agent;
(e) removal of the cleaving agent; and
(f) adding a capping group to any uncleaved 3′-O-azidomethyl groups.

According to a second aspect of the invention, there is provided the use of a kit in a method of nucleic acid synthesis, wherein said kit comprises a 3′-O-azidomethyl capping group as defined herein, optionally in combination with one or more components selected from: terminal deoxynucleotidyl transferase (TdT) or a functional equivalent or fragment thereof, an initiator sequence, one or more 3′-blocked nucleotide triphosphates, inorganic pyrophosphatase, such as purified, recombinant inorganic pyrophosphatase from Saccharomyces cerevisiae, a cleaving agent, an extension solution, a wash solution and/or a cleaving solution; further optionally together with instructions for use of the kit in accordance with the method as defined herein.

According to a further aspect of the invention, there is provided a capped nucleotide triphosphate selected from a compound of formula (I)^{a}, (II)^{a}, (III)^{a} or (IV)^{a}:

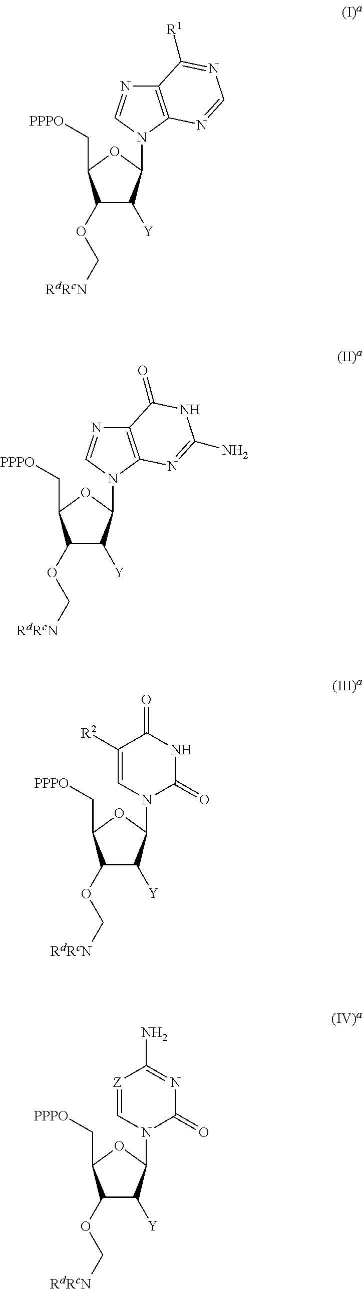

wherein
R¹ represents NR^{a}R^{b}, wherein R^{a} and R^{b} independently represent hydrogen or C₁₋₆ alkyl;
R² represents hydrogen, C₁₋₆ alkoxy, COH, COOH or C₁₋₆ alkyl optionally substituted by one or more OH or COOH groups;
Y represents hydrogen, hydroxyl or halogen;
Z represents CR⁴ or N, wherein R⁴ represents hydrogen, C₁₋₆ alkoxy, COH, COOH or C₁₋₆ alkyl optionally substituted by one or more OH or COOH groups; and
R^{c} and R^{d} together with the nitrogen atom to which they are attached join to form a triazole ring fused to one or more carbocyclic or heterocyclic ring systems, wherein said ring systems may be optionally substituted by any suitable functional groups, such as an amine, carboxylic acid, maleimide, one half of a binding pair (e.g. biotin), a fluorine containing moiety or a fluorescent moiety.

According to a further aspect of the invention, there is provided the use of an alkyne containing reagent as a 3′-O-azidomethyl capping group.

### BRIEF DESCRIPTION OF THE FIGURES

FIG. 1: Schematic of enzymatic DNA synthesis process. Starting from the top of the diagram, an immobilised strand of DNA with a deprotected 3′-end is exposed to an extension mixture composed of TdT, a base-specific 3′-blocked nucleotide triphosphate, inorganic pyrophosphatase to reduce the buildup of inorganic pyrophosphate, and appropriate buffers/salts for optimal enzyme activity and stability. The protein adds one protected nucleotide to the immobilised DNA strand (bottom of diagram). The extension mixture is then removed with wash mixture and optionally recycled. The immobilised (n+1) DNA strand is then washed with a cleavage mixture to cleave the 3′-protecting group, enabling reaction in the next cycle. In the cleavage mixture, denaturant may be present to disrupt any secondary structures. During this step, the temperature may be raised to assist in cleavage and disruption of secondary structures. The immobilised DNA is treated with wash mixture to remove leftover cleavage mixture. Steps 1-4 may be repeated with an appropriate nucleotide triphosphate until the desired oligonucleotide sequence is achieved.

FIG. 2: Overview of the capping method of the invention compared with the capping step found in phosphoramidite-based DNA synthesis.

FIG. 3: A capillary electrophoresis chromatogram showing a TdT-mediated addition of a 3′-O-azidomethyl thymidine triphosphate to a FAM-labeled DNA initiator. After the 60 min reaction, the products were incubated with DBCO-TAMRA, resulting in the conversion of 3′-O-azidomethyl containing DNA strands into the 1,2,3-triazole adduct. This reaction is evident due to the co-elution of a N+1 peak with signal in both the FAM and TAMRA channels. The solid star indicates a TAMRA fluorophore. On the molecular structure, B represents any nitrogenous base (such as A, T, C, G, U, hmC, mC, 8-oxo-G, etc.). X represents further nucleotides on the 5′-side.

FIG. 4: Simplified schematic representation of a column-based flow instrument used in DNA synthesis. A computer (302) controls two pumps and a solution mixing chamber (311). Pump 1 (304) selectively pumps extension solution (301), wash solution (305) or cleavage solution (310) into the mixing chamber. Pump 2 (306) selectively pumps a single 3′-blocked nucleotide triphosphate (TP) solution containing either 3′-blocked A(adenine)TP (303), T(thymine)TP (307), G(guanine)TP (308), or C(cytosine)TP (309) into the chamber. The computer controlled mixing chamber then passes appropriate solution ratios from pump 1 and pump 2 into a column based DNA synthesis chamber (312). A heating element (313) ensures that the DNA synthesis column remains at the necessary temperature for the synthesis process to take place. Upon exiting the DNA synthesis chamber, the reaction solution either enters a recycling vessel (314) for future use, a waste vessel (316) or moves on to a polymerase chain reaction (PCR) step (315) for amplification of the resultant DNA. PCR completion leads to the final product (317).

### DETAILED DESCRIPTION OF THE INVENTION

According to a first aspect of the invention, there is provided a method of nucleic acid synthesis, which comprises the steps of:
(a) providing an initiator sequence;
(b) adding a 3′-O-azidomethyl blocked nucleotide triphosphate to said initiator sequence in the presence of terminal deoxynucleotidyl transferase (TdT) or a functional equivalent or fragment thereof,
(c) removal of TdT;
(d) cleaving the 3′-O-azidomethyl group from the 3′-O-azidomethyl blocked nucleotide triphosphate in the presence of a cleaving agent;
(e) removal of the cleaving agent; and
(f) adding a capping group to any uncleaved 3′-O-azidomethyl groups.

The use described herein has significant advantages, such as the ability to rapidly produce long lengths of DNA while still maintaining a high accuracy and yield without using any toxic organic solvents.

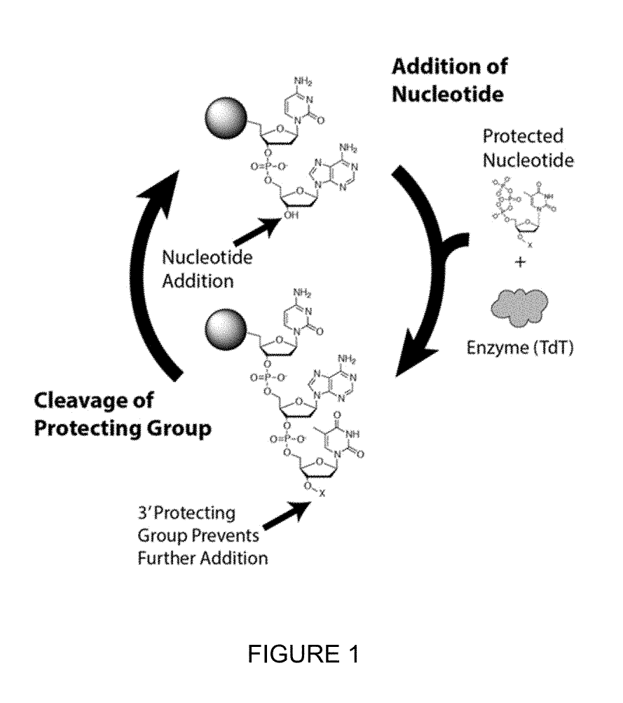

The 3′-O-azidomethyl represents one example of a protecting group which may be used to reversibly block nucleotide triphosphates in order to control the nucleic acid sequence during TdT-mediated coupling (see FIG. 1). During “deprotection,” the nucleotide added to a growing nucleic acid strand is deprotected and readied for the subsequent coupling step. This deprotection step is reported to be quantitative (Guo et al., Proc. Natl. Acad. Sci. 2008), but is not necessarily 100%. The present inventors have identified that a population of unreacted strands may survive through to the subsequent deprotection step, where they are deprotected to reveal the reactive hydroxyl group. This strand as a result becomes an N−1 mutant.

In addition to the potential to generate N−1 mutants from the deprotection step, the “coupling” step can also generate N−1 mutants if TdT fails to add a 3′-O-azidomethyl nucleotide triphosphate to each available free 3′-OH nucleic acid strand. Due to the iterative nature of DNA synthesis, errors are amplified geometrically (e.g., 99% coupling efficiency results in an effective yield of 0.99ⁿ, where n is the quantity of steps). Thus, small errors in each round result in loss of yield and control of nucleic acid sequence over time. Additionally, separating N−1 or more mutants from the desired sequence represents a significant challenge.

In order to overcome this problem, the invention makes use of 1,3-dipolar cycloaddition click chemistry. The advantages of the invention reduce the likelihood of (1) mutations resulting from unreacted nucleic acid strands during the coupling step (Capping 1) and (2) mutations resulting from 3′-O-azidomethyl groups that are not deprotected during the deprotection step (Capping 2).

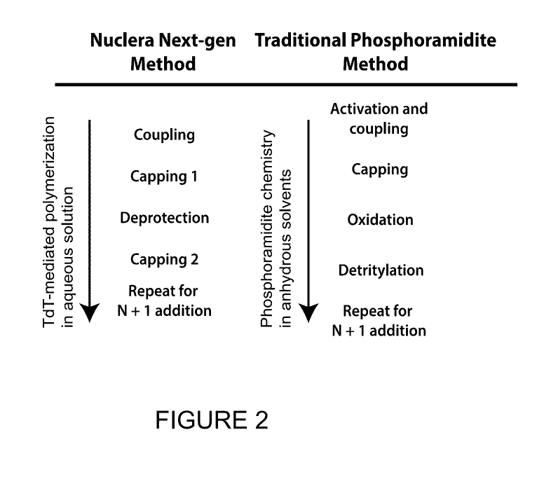

Traditional phosphoramidite DNA synthesis (see FIG. 2) utilises acetylation chemistry to cap unreacted hydroxyl groups, following the coupling step. Other capping methods involve phosphitylation. However, such chemistry is not suitable for a TdT-mediated synthesis method. Firstly, the commonly used acetylating reagents, acetic anhydride and N-methylimidazole (catalyst), are not stable under aqueous environments. Secondly, the 3′-O-azidomethyl, rather than the deprotected 3′-OH, needs to be blocked to prevent deletion mutants after the deprotection step. Finally, in phosphoramidite chemistry, nucleic acids are synthesised 3′ to 5′, whereas the method of the invention synthesises nucleic acids 5′ to 3′. As a result, a secondary alcohol (3′-OH) rather than a primary alcohol (5′-OH) would be exposed. A secondary alcohol is less reactive than a primary alcohol and thus more difficult to react in a quantitative fashion.

An issue encountered with acetylation capping chemistry is the removal of the acetyl cap during the post-synthesis ammonia cleavage/deprotection step. Removal of the cap regenerates the hydroxyl in failure sequences, rendering them active in biological processes such as enzymatic reactions. The cycloaddition product is stable under a wide range of conditions, and will remain present in failure sequences throughout the synthesis process of the invention.

In the coupling stage, if TdT fails to add a 3′-O-azidomethyl nucleotide triphosphate to a strand, that strand is left with a free 3′-OH. This 3′-OH can be trapped by incubation with TdT in a second coupling stage (called “Capping 1”) with a nucleotide triphosphate that does not contain a free 3′-OH function group for continued strand growth in subsequent cycles, such as 2′, 3′-dideoxy nucleotide triphosphate analogs. In an alternative embodiment, a 3′-azido or 3′-amino nucleotide triphosphate is added by TdT to an unreacted strand.

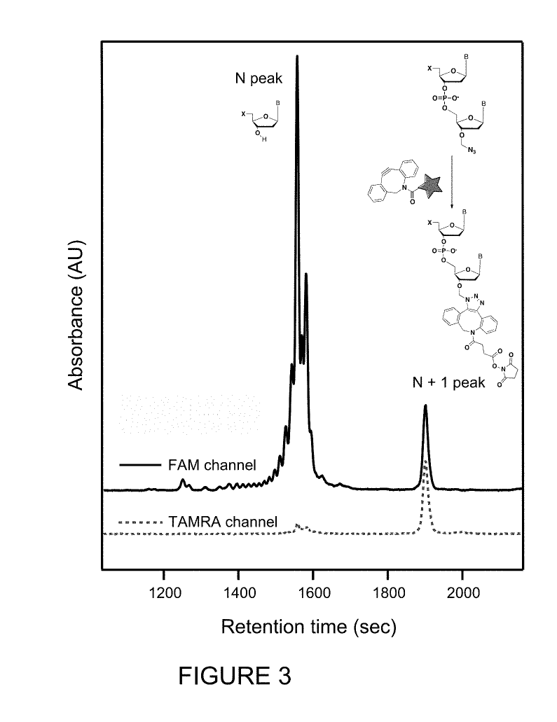

In the deprotecting stage, if tris(2-carboxyethyl)phosphine (TCEP) mediated deprotection fails to deprotect a 3′-O-azidomethyl group on a nucleic acid strand, the strand is left with an unreacted 3′-O-azidomethyl group. By reacting DNA strands containing a 3′-O-azidomethyl group with a capping group (called “Capping 2”), the inventors have surprisingly shown that the capping group serves as a suitable moiety to irreversibly cap a strand (see FIG. 3).

### Capping Groups

In one embodiment, the capping group is an irreversible capping group.

In one embodiment, the capping group is a dipolarophile. In a further embodiment, the dipolarophile is an alkyne. In a yet further embodiment, the alkyne is a strained alkyne.

In a yet further embodiment, the dipolarophile is dibenzocyclooctyne-amine (CAS Number: 1255942-06-3).

When the capping group is a dibenzocyclooctyne analogue, step (f) may typically comprise the reaction shown in Scheme 1:

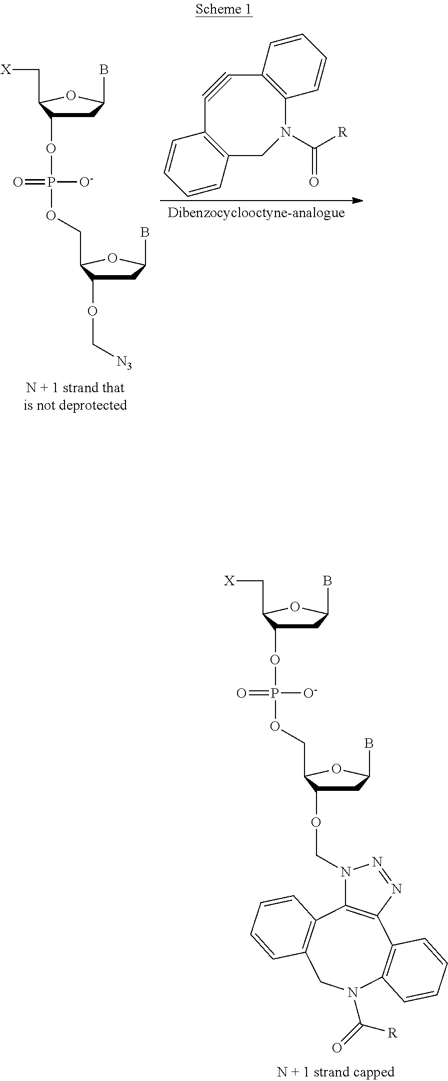

wherein X represents further nucleotides on the 5′-side. B represents any nitrogenous base, such as such as A, T, C, G, U, hmC, mC, 8-oxo-G, etc. R represents any functional group such as fluorophores, biotin, amine, carboxylic acid, maleimide, etc.

In this embodiment of the invention shown in Scheme 1, a dibenzocyclooctyne analogue is reacted with an N+1 strand that has failed to deprotect. The reaction results in a triazole adduct that is not labile to reducing agents, thereby irreversibly capping the strand.

In one embodiment, step (f) comprises an uncatalysed cycloaddition reaction.

In an alternative embodiment, step (f) comprises a cycloaddition reaction catalysed by a copper, ruthenium, or other transition metal-based catalyst.

In addition to capping deletion mutants, the capping group may serve as a purification handle to sequester deletion mutants. Thus, in one embodiment, the capping group comprises one half of a binding pair, such as biotin. Such coupling would allow capture of deletion mutants by exposure to avidin or streptavidin.

The capping group may also serve as a handle to facilitate liquid chromatographic separation of the product from deletion mutants. Such handles include fluorous tags (e.g.: CₙF₂ₙ₊₁) for use in fluorous HPLC. Thus, in one embodiment, the capping group comprises a fluorine-containing moiety.

In order to quantify the amount of deletion mutants generated over n cycles, a fluorescently-tagged capping group can be used. Thus, in one embodiment, the capping group comprises a fluorescent moiety. Such tagging allows for direct quantification via fluorescent spectroscopy to obtain the quantity of deletion mutants produced in the present DNA synthesis method as a result of deprotection failure.

In one embodiment, the 3′-O-azidomethyl blocked nucleotide triphosphate is selected from a compound of formula (I), (II), (III) or (IV):

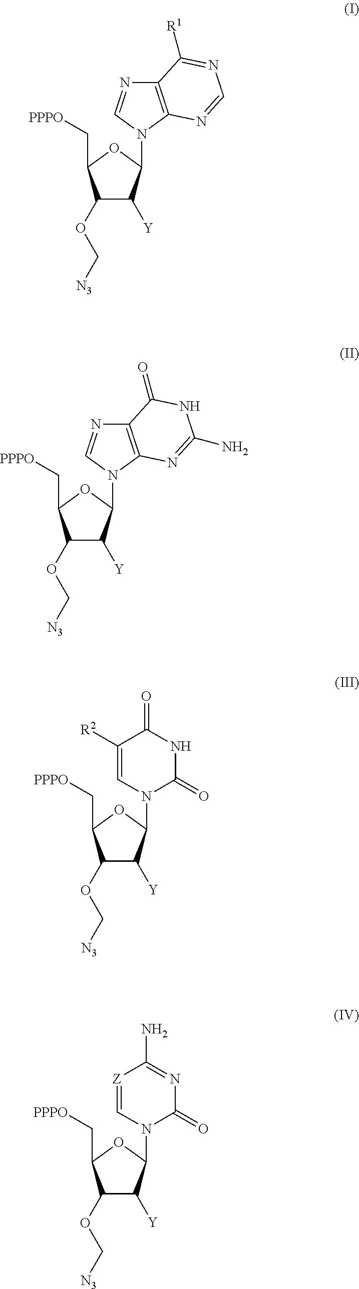

wherein
R¹ represents NR^{a}R^{b}, wherein R^{a} and R^{b} independently represent hydrogen or C₁₋₆ alkyl;
R² represents hydrogen, C₁₋₆ alkoxy, COH, COOH or C₁₋₆ alkyl optionally substituted by one or more OH or COOH groups;
Y represents hydrogen, hydroxyl or halogen; and
Z represents CR⁴ or N, wherein R⁴ represents hydrogen, C₁₋₆ alkoxy, COH, COOH or C₁₋₆ alkyl optionally substituted by one or more OH or COOH groups.

In one embodiment which may be mentioned, there is provided a compound of formula (I), (II), (III) or (IV) wherein

R¹ represents NR^{a}R^{b}, wherein R^{a} and R^{b} independently represent hydrogen or C₁₋₆ alkyl;
R² represents hydrogen, C₁₋₆ alkoxy, COH, COOH or C₁₋₆ alkyl optionally substituted by one or more OH or COOH groups;
Y represents hydrogen or hydroxyl; and
Z represents CR⁴ or N, wherein R⁴ represents C₁₋₆ alkoxy, COH, COOH or C₁₋₆ alkyl optionally substituted by one or more OH or COOH groups.

It will be understood that ‘PPP’ in the structures shown herein represents a triphosphate group.

References to the term ‘C₁₋₆ alkyl’ as used herein as a group or part of a group refers to a linear or branched saturated hydrocarbon group containing from 1 to 6 carbon atoms. Examples of such groups include methyl, ethyl, butyl, n-propyl, isopropyl and the like.

References to the term ‘C₁₋₆ alkoxy’ as used herein refer to an alkyl group bonded to oxygen via a single bond (i.e. R—O). Such references include those with straight and branched alkyl chains containing 1 to 6 carbon atoms, such as methoxy (or methyloxy), ethyloxy, n-propyloxy, iso-propyloxy, n-butyloxy and 2-methylpropyloxy.

References to the term ‘COOH’ or ‘CO₂H’ refer to a carboxyl group (or carboxy) which consists of a carbonyl (C═O) and a hydroxyl (O—H) group. References to the term ‘COH’ refer to a formyl group which consists of a carbonyl (C═O) group bonded to hydrogen.

The term ‘N₃’ (drawn structurally as —N═N⁺═N⁻) refers to an azido group.

In one embodiment, R^{a} and R^{b} both represent hydrogen (i.e. R¹ represents NH₂).

In an alternative embodiment, R^{a} represents hydrogen and R^{b} represents methyl (i.e. R¹ represents NHCH₃).

In one embodiment, R² represents hydrogen, methyl or methoxy. In a further embodiment, R² represents hydrogen. In an alternative embodiment, R² represents methyl. In a yet further alternative embodiment, R² represents methoxy.

In one embodiment, Y represents hydrogen.

In an alternative embodiment, Y represents hydroxyl.

In one embodiment, Z represents N.

In an alternative embodiment, Z represents CR⁴.

In one embodiment, R⁴ represents methoxy, COOH or COH. In a further embodiment, R⁴ represents methoxy. In an alternative embodiment, R⁴ represents COOH. In a yet further alternative embodiment, R⁴ represents COH.

In one embodiment, the 3′-blocked nucleotide triphosphate is selected from:

| Structure | Name | Example number |
|---|---|---|
| 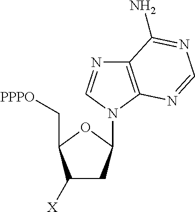 | Deoxyadenosine triphosphate | E1 |
| 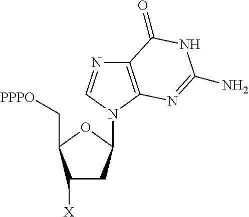 | Deoxyguanosine triphosphate | E2 |
| 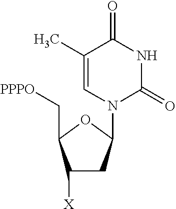 | Deoxythymidine triphosphate | E3 |
| 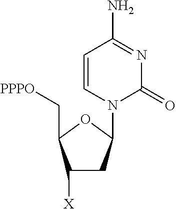 | Deoxycytidine triphosphate | E4 |
| 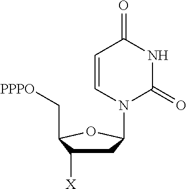 | 2′-deoxy-uridine triphosphate | E5 |
| 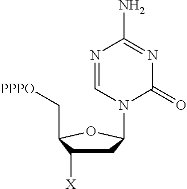 | 5-aza-2′-deoxy-cytidine- triphosphate | E6 |
| 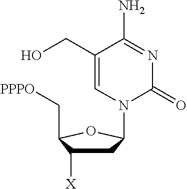 | 5-hydroxymethyl- deoxycytidine triphosphate | E7 |
| 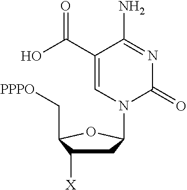 | 5-carboxy-deoxycytidine triphosphate | E8 |
| 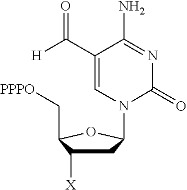 | 5-formyl-deoxycytidine triphosphate | E9 |
| 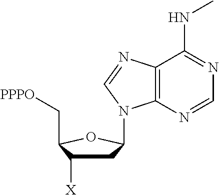 | N6-methyladenosine triphosphate | E10 |
| 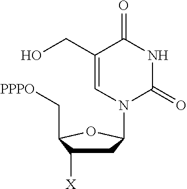 | 5-hydroxymethyl-deoxy- uridine triphosphate | E11 |

wherein ‘X’ represents —O—CH₂—N₃.

According to a further aspect of the invention, there is provided a capped nucleotide triphosphate selected from a compound of formula (I)^{a}, (II)^{a}, (III)^{a} or (IV)^{a}:

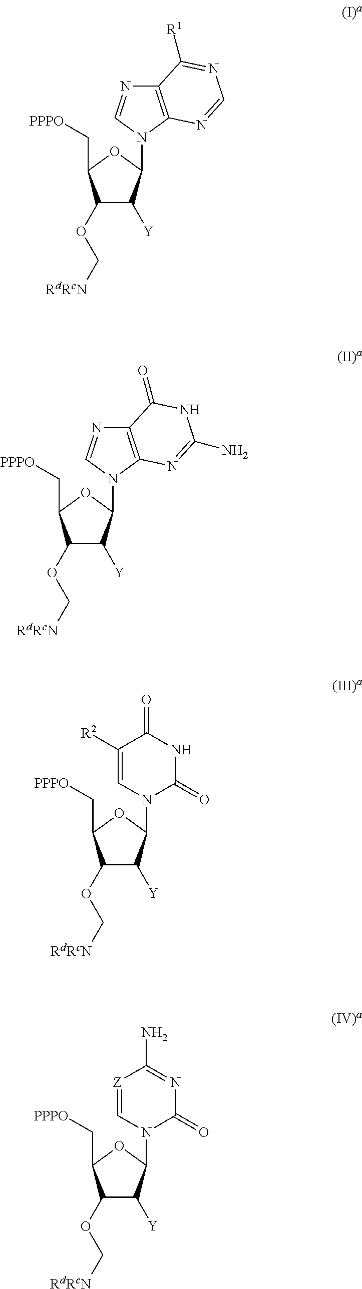

wherein
R¹ represents NR^{a}R^{b}, wherein R^{a} and R^{b} independently represent hydrogen or C₁₋₆ alkyl;
R² represents hydrogen, C₁₋₆ alkoxy, COH, COOH or C₁₋₆ alkyl optionally substituted by one or more OH or COOH groups;
Y represents hydrogen, hydroxyl or halogen;
Z represents CR⁴ or N, wherein R⁴ represents hydrogen, C₁₋₆ alkoxy, COH, COOH or C₁₋₆ alkyl optionally substituted by one or more OH or COOH groups; and
R^{c} and R^{d} together with the nitrogen atom to which they are attached join to form a triazole ring fused to one or more carbocyclic or heterocyclic ring systems, wherein said ring systems may be optionally substituted by any suitable functional groups, such as an amine, carboxylic acid, maleimide, one half of a binding pair (e.g. biotin), a fluorine containing moiety or a fluorescent moiety.

In one embodiment which may be mentioned, there is provided a compound of formula (I)^{a}, (II)^{a}, (III)^{a} or (IV)^{a} wherein

R¹ represents NR^{a}R^{b}, wherein R^{a} and R^{b} independently represent hydrogen or C₁₋₆ alkyl;
R² represents hydrogen, C₁₋₆ alkoxy, COH, COOH or C₁₋₆ alkyl optionally substituted by one or more OH or COOH groups;
Y represents hydrogen or hydroxyl;
Z represents CR⁴ or N, wherein R⁴ represents C₁₋₆ alkoxy, COH, COOH or C₁₋₆ alkyl optionally substituted by one or more OH or COOH groups; and
R^{c} and R^{d} together with the nitrogen atom to which they are attached join to form a triazole ring fused to one or more carbocyclic or heterocyclic ring systems, wherein said ring systems may be optionally substituted by any suitable functional groups, such as an amine, carboxylic acid, maleimide, one half of a binding pair (e.g. biotin), a fluorine containing moiety or a fluorescent moiety.

In one embodiment, —NR^{c}R^{d} represents a group of formula (V):

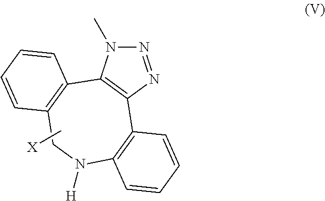

wherein X represents one or more suitable functional groups, such as an amine, carboxylic acid, maleimide, one half of a binding pair (e.g. biotin), a fluorine containing moiety or a fluorescent moiety.

According to a further aspect of the invention, there is provided the use of an alkyne containing reagent as a 3′-O-azidomethyl capping group.

In one embodiment, the alkyne containing reagent is selected from a compound of formula (VI):

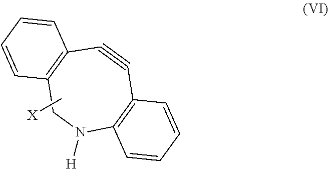

wherein X represents one or more suitable functional groups, such as an amine, carboxylic acid, maleimide, one half of a binding pair (e.g. biotin), a fluorine containing moiety or a fluorescent moiety. When the alkyne containing reagent comprises a compound of formula (VI) the process of capping a 3′-O-azidomethyl group typically comprises a cycloaddition reaction as described hereinbefore in Scheme 1.

In an alternative embodiment, the alkyne containing reagent is selected from a compound of formula (VII):

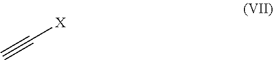

wherein X is as defined hereinbefore. When the alkyne containing reagent comprises a compound of formula (VII) the process of capping a 3′-O-azidomethyl group typically comprises a cycloaddition reaction in accordance with procedures known to the skilled person, such as a cycloaddition reaction catalysed by a transition metal-based catalyst.

According to a further aspect of the invention, there is provided a 3′-O-azidomethyl capping group selected from a compound of formula (VI) or (VII).

### Terminal Deoxynucleotidyl Transferase (TdT) Enzymes

References herein to terminal deoxynucleotidyl transferase (TdT) enzyme include references to purified and recombinant forms of said enzyme. It will be appreciated that references herein to “homology” are to be understood as meaning the similarity between two protein sequences, e.g.: SEQ ID NO: X and SEQ ID NO: Y, which is calculated by addition of the the common amino acids between aligned sequences SEQ ID NO: X and SEQ ID NO: Y, divided by the longer length of either SEQ ID NO: X or SEQ ID NO: Y, expressed as a percentage.

In one embodiment, the terminal deoxynucleotidyl transferase (TdT) is a natural TdT or non-natural TdT or a functional equivalent or fragment thereof.

It will be understood that the term ‘functional equivalent’ refers to the polypeptides which are different to the exact sequence of a TdT (such as Bos taurus TdT), but can perform the same function, i.e. catalyse the addition of a nucleotide triphosphate onto the 3′-end of a DNA strand in a template dependent manner.

In one embodiment, the terminal deoxynucleotidyl transferase (TdT) enzyme comprises an amino acid sequence selected from any one of SEQ ID NOS: 1 to 5 and 8 or a functional equivalent or fragment thereof having at least 20% sequence homology to said amino acid sequence.

In a further embodiment, the terminal deoxynucleotidyl transferase (TdT) enzyme comprises an amino acid sequence selected from SEQ ID NO: 1. The amino acid sequence of SEQ ID NO. 1 is the terminal deoxynucleotidyl transferase (TdT) sequence from Sarcophilus harrisii (UniProt: G3VQ55). Sarcophilus harrisii (also known as the Tasmanian devil) is a carnivorous marsupial of the family Dasyuridae, now found in the wild only on the Australian island state of Tasmania.

In a further embodiment, the terminal deoxynucleotidyl transferase (TdT) enzyme comprises an amino acid sequence selected from SEQ ID NO: 2. The amino acid sequence of SEQ ID NO. 2 is the terminal deoxynucleotidyl transferase (TdT) sequence from Lepisosteus oculatus (UniProt: W5MK82). Lepisosteus oculatus (also known as the spotted gar) is a primitive freshwater fish of the family Lepisosteidae, native to North America from the Lake Erie and southern Lake Michigan drainages south through the Mississippi River basin to Gulf Slope drainages, from lower Apalachicola River in Florida to Nueces River in Texas, USA.

In a further embodiment, the terminal deoxynucleotidyl transferase (TdT) enzyme comprises an amino acid sequence selected from SEQ ID NO: 3. The amino acid sequence of SEQ ID NO. 3 is the terminal deoxynucleotidyl transferase (TdT) sequence from Chinchilla lanigera (NCBI Reference Sequence: XP_005407631.1; http://www.ncbi.nlm.nih.qoviproteini533189443). Chinchilla lanigera (also known as the long-tailed chinchilla, Chilean, coastal, common chinchilla, or lesser chinchilla), is one of two species of rodents from the genus Chinchilla, the other species being Chinchilla chinchilla.

In a further embodiment, the terminal deoxynucleotidyl transferase (TdT) enzyme comprises an amino acid sequence selected from SEQ ID NO: 4. The amino acid sequence of SEQ ID NO. 4 is the terminal deoxynucleotidyl transferase (TdT) sequence from Otolemur garnettii (UniProt: A4PCE6). Otolemur garnettii (also known as the northern greater galago, Garnett's greater galago or small-eared greater galago), is a nocturnal, arboreal primate endemic to Africa.

In a further embodiment, the terminal deoxynucleotidyl transferase (TdT) enzyme comprises an amino acid sequence selected from SEQ ID NO: 5. The amino acid sequence of SEQ ID NO. 5 is the terminal deoxynucleotidyl transferase (TdT) sequence from Sus scrofa (UniProt: F1SBG2). Sus scrofa (also known as the wild boar, wild swine or Eurasian wild pig) is a suid native to much of Eurasia, North Africa and the Greater Sunda Islands.

In an alternative embodiment, the terminal deoxynucleotidyl transferase (TdT) enzyme comprises an amino acid sequence selected from Bos taurus (UniProt: P06526). Bos taurus (also known as cattle, or colloquially cows) are the most common type of large domesticated ungulates. They are a prominent modern member of the subfamily Bovinae, are the most widespread species of the genus Bos.

In a further embodiment, the terminal deoxynucleotidyl transferase (TdT) enzyme comprises an amino acid sequence selected from SEQ ID NO: 8. The amino acid sequence of SEQ ID NO: 8 is a variant of SEQ ID NO: 2 which has been engineered for improved activity by alteration of the amino acid sequence.

In a further embodiment, the terminal deoxynucleotidyl transferase (TdT) enzyme comprises an amino acid sequence selected from SEQ ID NOS: 1, 2 or 8.

In an alternative embodiment, the terminal deoxynucleotidyl transferase (TdT) enzyme comprises an amino acid sequence selected from a modified derivative of SEQ ID NO: 6 (i.e. a non-natural, mutated derivative of SEQ ID NO: 6). The amino acid sequence of SEQ ID NO: 6 is the terminal deoxynucleotidyl transferase (TdT) sequence from Bos taurus (UniProt: P06526). Bos taurus (also known as cattle, or colloquially cows) are the most common type of large domesticated ungulates. They are a prominent modern member of the subfamily Bovinae, are the most widespread species of the genus Bos.

References herein to ‘fragment’ include, for example, functional fragments with a C-terminal truncation, or with an N-terminal truncation. Fragments are suitably greater than 10 amino acids in length, for example greater than 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 amino acids in length.

In one embodiment, the terminal deoxynucleotidyl transferase (TdT) has at least 25% homology with the TdTs described herein, such as at least 30%, 40%, 50%, 55%, 60%, 65%, 70%, 75%, 80%, 85%, 90%, 95%, 96%, 97%, 98% or 99% homology.

### Nucleic Acid Synthesis

References herein to a “method of nucleic acid synthesis” include methods of synthesising lengths of DNA (deoxyribonucleic acid) or RNA (ribonucleic acid) wherein a strand of nucleic acid (n) is extended by adding a further nucleotide (n+1). In one embodiment, the nucleic acid is DNA. In an alternative embodiment, the nucleic acid is RNA.

References herein to “method of DNA synthesis” refer to a method of DNA strand synthesis wherein a DNA strand (n) is extended by adding a further nucleotide (n+1). The method described herein provides a novel use of the terminal deoxynucleotidyl transferases of the invention and 3′-blocked nucleotide triphosphates to sequentially add nucleotides in de novo DNA strand synthesis which has several advantages over the DNA synthesis methods currently known in the art.

It will be understood that steps (b) to (f) of the method may be repeated multiple times to produce a DNA or RNA strand of a desired length. Therefore, in one embodiment, greater than 1 nucleotide is added to the initiator sequence, such as greater than 5, 10, 20, 30, 40, 50, 60, 70, 80, 90, 100, 110 or 120 nucleotides are added to the initiator sequence by repeating steps (b) to (f). In a further embodiment, greater than 200 nucleotides are added, such as greater than 300, 400, 500, 600, 700, 800, 900, 1000, 1250, 1500, 1750, 2000, 2250, 2500, 2750, 3000, 4000, 5000, 6000, 7000, 8000, 9000 or 10000 nucleotides.

### 3′-Blocked Nucleotide Triphosphates

References herein to ‘nucleotide triphosphates’ refer to a molecule containing a nucleoside (i.e. a base attached to a deoxyribose or ribose sugar molecule) bound to three phosphate groups. Examples of nucleotide triphosphates that contain deoxyribose are: deoxyadenosine triphosphate (dATP), deoxyguanosine triphosphate (dGTP), deoxycytidine triphosphate (dCTP) or deoxythymidine triphosphate (dTTP). Examples of nucleotide triphosphates that contain ribose are: adenosine triphosphate (ATP), guanosine triphosphate (GTP), cytidine triphosphate (CTP) or uridine triphosphate (UTP). Other types of nucleosides may be bound to three phosphates to form nucleotide triphosphates, such as artificial nucleosides.

Therefore, references herein to ‘3′-O-azidomethyl blocked nucleotide triphosphates’ refer to nucleotide triphosphates (e.g., dATP, dGTP, dCTP or dTTP) which have an additional azidomethyl group (i.e. —O—CH₂—N₃) on the 3′ end which prevents further addition of nucleotides, i.e., by replacing the 3′—OH group with a 3′-O-azidomethyl protecting group.

It will be understood that references herein to ‘3′-block’, ‘3′-blocking group’ or ‘3′-protecting group’ refer to the group attached to the 3′ end of the nucleotide triphosphate which prevents further nucleotide addition. The present method uses reversible 3′-protecting groups (i.e. 3′-O-azidomethyl) which can be removed by cleavage to allow the addition of further nucleotides.

### Cleaving Agent

References herein to ‘cleaving agent’ refer to a substance which is able to cleave the 3′-O-azidomethyl blocking group from the 3′-blocked nucleotide triphosphate.

The 3′-O-azidomethyl blocking group may be quantitatively removed in aqueous solution with documented compounds which may be used as cleaving agents (for example, see: Wuts, P. G. M. & Greene, T. W. (2012) 4th Ed., John Wiley & Sons; Hutter, D. et al. (2010) Nucleosides Nucleotides Nucleic Acids 29, 879-895; EP 1560838 and U.S. Pat. No. 7,795,424).

In one embodiment, the cleaving agent is a chemical cleaving agent. In an alternative embodiment, the cleaving agent is an enzymatic cleaving agent.

In one embodiment, tris(2-carboxyethyl)phosphine (TCEP) can be used to cleave a 3′-O-azidomethyl group.

In one embodiment, the cleaving agent is added in the presence of a cleavage solution comprising a denaturant, such as urea, guanidinium chloride, formamide or betaine. The addition of a denaturant has the advantage of being able to disrupt any undesirable secondary structures in the DNA. In a further embodiment, the cleavage solution comprises one or more buffers. It will be understood by the person skilled in the art that the choice of buffer is dependent on the exact cleavage chemistry and cleaving agent required.

### Initiator Sequences

References herein to an ‘initiator sequence’ refer to a short oligonucleotide with a free 3′-end which the 3′-O-azidomethyl blocked nucleotide triphosphate can be attached to. In one embodiment, the initiator sequence is a DNA initiator sequence. In an alternative embodiment, the initiator sequence is an RNA initiator sequence.

References herein to a ‘DNA initiator sequence’ refer to a small sequence of DNA which the 3′-blocked nucleotide triphosphate can be attached to, i.e. DNA will be synthesised from the end of the DNA initiator sequence.

In one embodiment, the initiator sequence is between 5 and 50 nucleotides long, such as between 5 and 30 nucleotides long (i.e. between 10 and 30), in particular between 5 and 20 nucleotides long (i.e., approximately 20 nucleotides long), more particularly 5 to 15 nucleotides long, for example 10 to 15 nucleotides long, especially 12 nucleotides long.

In one embodiment, the initiator sequence has the following sequence: 5′-CGTTAACATATT-3′ (SEQ ID NO: 7).

In one embodiment, the initiator sequence is single-stranded. In an alternative embodiment, the initiator sequence is double-stranded. It will be understood by persons skilled in the art that a 3′-overhang (i.e., a free 3′-end) allows for efficient addition.

In one embodiment, the initiator sequence is immobilised on a solid support. This allows TdT and the cleaving agent to be removed (in steps (c) and (e), respectively) without washing away the synthesised nucleic acid. The initiator sequence may be attached to a solid support stable under aqueous conditions so that the method can be easily performed via a flow setup or microarray setup.

In one embodiment, the initiator sequence is immobilised on a solid support via a reversible interacting moiety, such as a chemically-cleavable linker, an antibody/immunogenic epitope, a biotin/biotin binding protein (such as avidin or streptavidin), or glutathione-GST tag. Therefore, in a further embodiment, the method additionally comprises extracting the resultant nucleic acid by removing the reversible interacting moiety in the initiator sequence, such as by incubating with proteinase K.

In a further embodiment, the initiator sequence is immobilised on a solid support via a chemically-cleavable linker, such as a disulfide, allyl, or azide-masked hemiaminal ether linker. Therefore, in one embodiment, the method additionally comprises extracting the resultant nucleic acid by cleaving the chemical linker through the addition of tris(2-carboxyethyl)phosphine (TCEP) or dithiothreitol (DTT) for a disulfide linker; palladium complexes for an allyl linker; or TCEP for an azide-masked hemiaminal ether linker.

In one embodiment, the resultant nucleic acid is extracted and amplified by polymerase chain reaction using the nucleic acid bound to the solid support as a template. The initiator sequence could therefore contain an appropriate forward primer sequence and an appropriate reverse primer could be synthesised.

In an alternative embodiment, the immobilised initiator sequence contains at least one restriction site. Therefore, in a further embodiment, the method additionally comprises extracting the resultant nucleic acid by using a restriction enzyme.

The use of restriction enzymes and restriction sites to cut nucleic acids in a specific location is well known in the art. The choice of restriction site and enzyme can depend on the desired properties, for example whether ‘blunt’ or ‘sticky’ ends are required. Examples of restriction enzymes include: AluI, BamHI, EcoRI, EcoRII, EcoRV, HaeII, HgalI, HindIII, HinfI, NotI, PstI, PvuII, SalI, Sau3A, ScaI, SmaI, TaqI and XbaI.

### Nucleic Acid Synthesis Method

In one embodiment, the terminal deoxynucleotidyl transferase (TdT) of the invention is added in the presence of an extension solution comprising one or more buffers (e.g., Tris or cacodylate), one or more salts (e.g., Na⁺, K⁺, Mg²⁺, Mn²⁺, Cu²⁺, Zn²⁺, Co²⁺, etc., all with appropriate counterions, such as Cl⁻) and inorganic pyrophosphatase (e.g., the Saccharomyces cerevisiae homolog). It will be understood that the choice of buffers and salts depends on the optimal enzyme activity and stability.

The use of an inorganic pyrophosphatase helps to reduce the build-up of pyrophosphate due to nucleotide triphosphate hydrolysis by TdT. Therefore, the use of an inorganic pyrophosphatase has the advantage of reducing the rate of (1) backwards reaction and (2) TdT strand dismutation. Thus, according to a further aspect of the invention, there is provided the use of inorganic pyrophosphatase in a method of nucleic acid synthesis. In one embodiment, the inorganic pyrophosphatase comprises purified, recombinant inorganic pyrophosphatase from Saccharomyces cerevisiae.

In one embodiment, step (b) is performed at a pH range between 5 and 10. Therefore, it will be understood that any buffer with a buffering range of pH 5-10 could be used, for example cacodylate, Tris, HEPES or Tricine, in particular cacodylate or Tris.

In one embodiment, step (d) is performed at a temperature less than 99° C., such as less than 95° C., 90° C., 85° C., 80° C., 75° C., 70° C., 65° C., 60° C., 55° C., 50° C., 45° C., 40° C., 35° C., or 30° C. It will be understood that the optimal temperature will depend on the cleavage agent utilised. The temperature used helps to assist cleavage and disrupt any secondary structures formed during nucleotide addition.

In one embodiment, steps (c) and (e) are performed by applying a wash solution. In one embodiment, the wash solution comprises the same buffers and salts as used in the extension solution described herein. This has the advantage of allowing the wash solution to be collected after step (c) and recycled as extension solution in step (b) when the method steps are repeated.

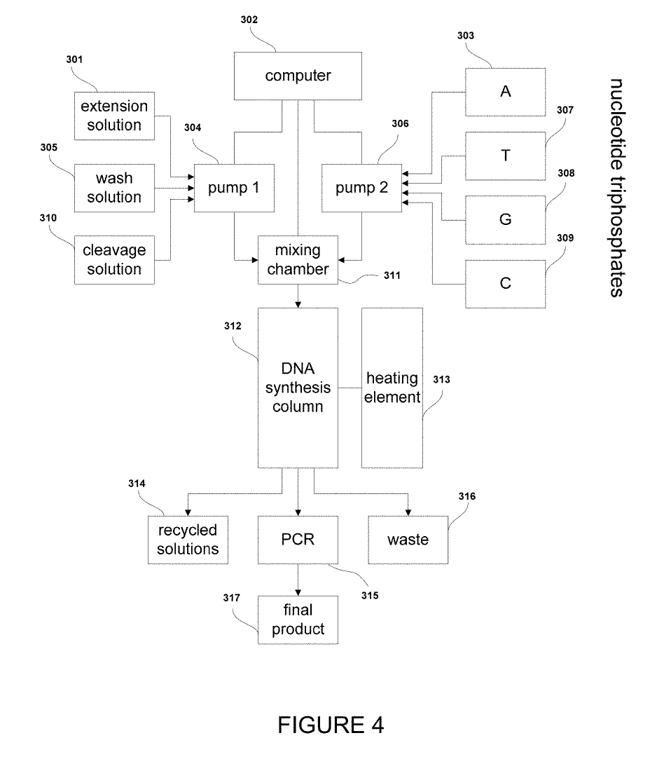

In one embodiment, the method is performed within a flow instrument as shown in FIG. 4, such as a microfluidic or column-based flow instrument. The method described herein can easily be performed in a flow setup which makes the method simple to use. It will be understood that examples of commercially available DNA synthesisers (e.g., MerMade 192E from BioAutomation or H-8 SE from K&A) may be optimised for the required reaction conditions and used to perform the method described herein.

In one embodiment, the method is performed on a plate or microarray setup. For example, nucleotides may be individually addressed through a series of microdispensing nozzles using any applicable jetting technology, including piezo and thermal jets. This highly parallel process may be used to generate hybridization microarrays and is also amenable to DNA fragment assembly through standard molecular biology techniques.

In one embodiment, the method additionally comprises amplifying the resultant nucleic acid. Methods of DNA/RNA amplification are well known in the art. For example, in a further embodiment, the amplification is performed by polymerase chain reaction (PCR). This step has the advantage of being able to extract and amplify the resultant nucleic acid all in one step.

The template independent nucleic acid synthesis method described herein has the capability to add a nucleic acid sequence of defined composition and length to an initiator sequence. Therefore, it will be understood by persons skilled in the art, that the method described herein may be used as a novel way to introduce adapter sequences to a nucleic acid library.

If the initiator sequence is not one defined sequence, but instead a library of nucleic acid fragments (for example generated by sonication of genomic DNA, or for example messenger RNA) then this method is capable of de novo synthesis of ‘adapter sequences’ on every fragment. The installation of adapter sequences is an integral part of library preparation for next-generation library nucleic acid sequencing methods, as they contain sequence information allowing hybridisation to a flow cell/solid support and hybridisation of a sequencing primer.

Currently used methods include single stranded ligation, however this technique is limited because ligation efficiency decreases strongly with increasing fragment length. Consequently, current methods are unable to attach sequences longer than 100 nucleotides in length. Therefore, the method described herein allows for library preparation in an alternative fashion to that which is currently possible.

Therefore, in one embodiment, an adapter sequence is added to the initiator sequence. In a further embodiment, the initiator sequence may be a nucleic acid from a library.

### Kits

According to a further aspect of the invention, there is provided the use of a kit in a method of nucleic acid synthesis, wherein said kit comprises a 3′-O-azidomethyl capping group as defined herein, optionally in combination with one or more components selected from: terminal deoxynucleotidyl transferase (TdT) or a functional equivalent or fragment thereof, an initiator sequence, one or more 3′-blocked nucleotide triphosphates, inorganic pyrophosphatase, such as purified, recombinant inorganic pyrophosphatase from Saccharomyces cerevisiae, a cleaving agent, an extension solution, a wash solution and/or a cleaving solution; further optionally together with instructions for use of the kit in accordance with the method as defined herein.

The following studies and protocols illustrate embodiments of the methods described herein:

A single-stranded DNA initiator labeled with a 5′-FAM tag was incubated with (1) 15 U Bos taurus TdT, (2) required salts (50 mM potassium acetate, 20 mM tris acetate pH 7.9, 1 mM cobalt chloride), and (3) 3′-O-azidomethyl TTP at 37° C. for 60 min. The 3′-blocked nucleotide triphosphate was at a concentration of 1 mM and the DNA initiator at 200 nM. The reaction was then stopped with EDTA and exposed to 30 μM DBCO-PEG4-TAMRA for 30 min. The reaction was then analysed in the FAM (solid line) and TAMRA (dotted line) channels by capillary electrophoresis, as shown in FIG. 3. The successful addition of DBCO-PEG4-TAMRA (illustrated by a reaction schematic presented in FIG. 3) is evident by the appearance of a DNA species with (1) higher retention compared to the N peak and (2) comparable signal in both the FAM and TAMRA emission channels, which indicates that the DNA species contains both FAM and TAMRA fluorophores (N+1 peak in FIG. 3).

The cycloaddition of an azide to an alkyne results in a 1,2,3-triazole that is stable to reducing agents, such as β-mercaptoethanol (BME), DTT, and TCEP. Thus, treatment of a nucleic acid strand containing a 3′-O-azidomethyl protecting group with an alkyne species, such as a DBCO analogue, will render the nucleic acid strand irreversibly blocked in a DNA synthesis method and incapable of further extension.

1. A method of nucleic acid synthesis, which comprises the steps of:
(a) providing an initiator sequence;
(b) adding a 3′-O-azidomethyl blocked nucleotide triphosphate to said initiator sequence in the presence of terminal deoxynucleotidyl transferase (TdT) or a functional equivalent or fragment thereof,
(c) removal of TdT;
(d) cleaving the 3′-O-azidomethyl group from the 3′-O-azidomethyl blocked nucleotide triphosphate in the presence of a cleaving agent;
(e) removal of the cleaving agent; and
(f) adding a capping group to any uncleaved 3′-O-azidomethyl groups.

2. The method as defined in claim 1, wherein the capping group is an irreversible capping group.

3. The method as defined in claim 1, wherein the capping group is a dipolarophile.

4. The method as defined in claim 3, wherein the dipolarophile is an alkyne, such as a strained alkyne.

5. The method as defined in claim 3, wherein the dipolarophile is dibenzocyclooctyne-amine.

6. The method as defined in claim 1, wherein step (f) comprises an uncatalysed cycloaddition reaction.

7. The method as defined in claim 1, wherein step (f) comprises a cycloaddition reaction catalysed by a copper or ruthenium-based catalyst.

8. The method as defined in claim 1, wherein the capping group comprises one half of a binding pair, such as biotin.

9. The method as defined in claim 1, wherein the capping group comprises a fluorine containing moiety.

10. The method as defined in claim 1, wherein the capping group comprises a fluorescent moiety.

11. The method as defined in claim 1, wherein the 3′-O-azidomethyl blocked nucleotide triphosphate is selected from a compound of formula (I), (II), (III) or (IV): 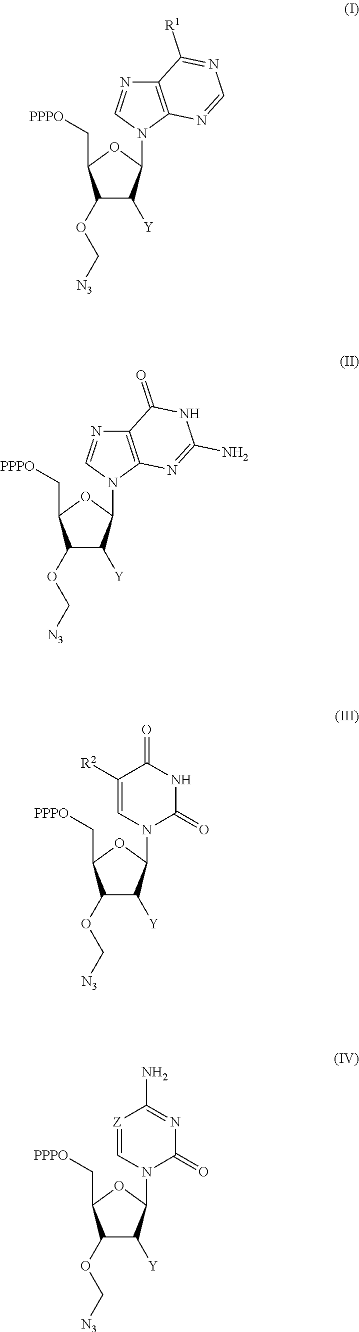
wherein:
R¹ represents NR^{a}R^{b}, wherein R^{a} and R^{b} independently represent hydrogen or C₁₋₆ alkyl;
R² represents hydrogen, C₁₋₆ alkoxy, COH, COOH or C₁₋₆ alkyl optionally substituted by one or more OH or COOH groups;
Y represents hydrogen, hydroxyl or halogen; and
Z represents CR⁴ or N, wherein R⁴ represents hydrogen, C₁₋₆ alkoxy, COH, COOH or C₁₋₆ alkyl optionally substituted by one or more OH or COOH groups.

12. The method as defined in claim 1, wherein the terminal deoxynucleotidyl transferase (TdT) enzyme comprises: an amino acid sequence selected from any one of SEQ ID NOS: 1 to 5 and 8 or a functional equivalent or fragment thereof having at least 20% sequence homology to said amino acid sequence; or a non-natural, mutated derivative of SEQ ID NO: 6.

13. The method as defined in claim 1, wherein greater than 1 nucleotide is added by repeating steps (b) to (f).

14. The method as defined in, claim 1, where the method is undertaken using a kit, said kit comprising a 3′-O-azidomethyl capping group, optionally in combination with one or more components selected from: terminal deoxynucleotidyl transferase (TdT) or a functional equivalent or fragment thereof, an initiator sequence, one or more 3′-blocked nucleotide triphosphates, inorganic pyrophosphatase, such as purified, recombinant inorganic pyrophosphatase from Saccharomyces cerevisiae, a cleaving agent, an extension solution, a wash solution and/or a cleaving solution; further optionally together with instructions for use of the kit.

15. A capped nucleotide triphosphate selected from a compound of formula (I)^{a}, (II)^{a}, (III)^{a}, or (IV)^{a}: 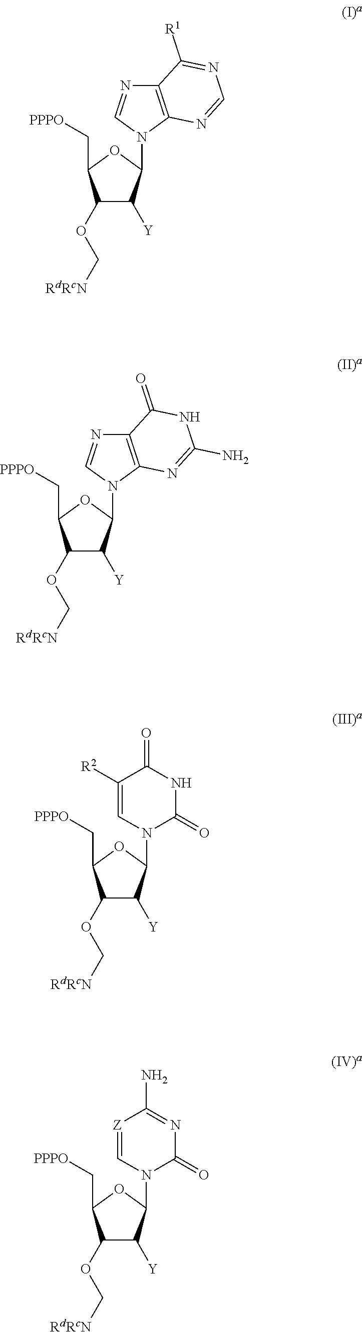
wherein
R¹ represents NR^{a}R, wherein R^{a} and R independently represent hydrogen or C₁₋₆alkyl;
R² represents hydrogen, C₁₋₆ alkoxy, COH, COOH or C₁₋₆ alkyl optionally substituted by or more OH or COOH groups;
Y represents hydrogen, hydroxyl or halogen;
Z represents CR⁴ or N, wherein R⁴ represents hydrogen, C₁₋₆ alkoxy, COH, COOH or C₁₋₆ alkyl optionally substituted by one or more OH or COOH groups; and
R^{c} and R^{d} together with the nitrogen atom to which they are attached join to form a triazole ring fused to one or more carbocyclic or heterocyclic ring systems, wherein said ring systems may be optionally substituted by any suitable functional groups, such as an amine, carboxylic acid, maleimide, one half of a binding pair, biotin, a fluorine containing moiety or a fluorescent moiety.

16. The capped nucleotide triphosphate as defined in claim 15, wherein —NR^{c}R^{d} represents a group of formula (V): 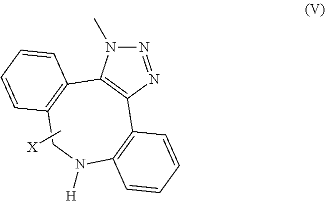
wherein:
X represents one or more suitable functional groups, such as an amine, carboxylic acid, maleimide, one half of a binding pair, biotin, a fluorine containing moiety or a fluorescent moiety.

17. A method of capping a 3′-O-azidomethyl group, comprising contacting an alkyne containing reagent with a 3′-O-azidomethyl.

18. The method as defined in claim 17, wherein the alkyne containing reagent is selected from a compound of formula (VI): 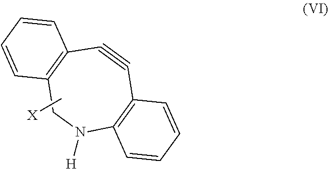
wherein:
X represents one or more suitable functional groups, such as an amine, carboxylic acid, maleimide, one half of a binding pair, biotin, a fluorine containing moiety or a fluorescent moiety.

19. The method as defined in claim 17, wherein the alkyne containing reagent is selected from a compound of formula (VII): 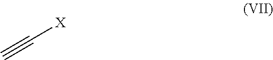
wherein
X represents one or more suitable functional groups, such as an amine, carboxylic acid, maleimide, one half of a binding pair, biotin, a fluorine containing moiety or a fluorescent moiety.

20. A 3′-O-azidomethyl capping group selected from a compound of formula (VI) or (VII).


SEQUENCE LISTING (8 sequences)

SEQ ID NO: 1 - protein, 517 aa, Sarcophilus harrisii
MHRIRTTDSDHGKKRQKKMDAISSKLYEIKFHEFVLFILEKKMGATRRTFLMDLARKKGFRVESELSNSVTHIVAENNSGSDVLAWLEAHKLETTAHFELLDVSWLIECMKVGKPVDTKGKYQLVESSIASANPDPNEGMLKIQSPAMNAISPYACQRRTTLNNHNQRFTDAFEILAKNYEFRENHGHCLTFLRATSVLKCLPFAIVSMKDAEGLPWIGDEVKGIMEEIIEDGQSLEVQAVLNDERYQAFKLFTSVFGVGLKTAEKWYRMGFRTLSKIQSDKSLKFTKMQKAGFLYYEDLISCVSKAEADAVSLIVKEAVWTFLPDALITITGGFRRGKEFGHDVDFLITSPGGEKEQVDQLLQKVTNLWEKQGLLLYYDLMESTFEDLKLPSRKVDALDHFQKCFLILKLYCQRGDRSKWEGPEGSNGLQTKNWKAIRVDLVVCPYDRYAYALLGWSGSRQFERDLRRYATHEKKMMLDNHALYDKTKRTFLKAESEEEIFSHLGLEYIEPWERNA

SEQ ID NO: 2 - protein, 494 aa, Lepisosteus oculatus
MLHIPIFPPIKKRQKLPESRNSCKYEVKFSEVAIFLVERKMGSSRRKFLTNLARSKGFRIEDVLSDAVTHVVAEDNSADELWQWLQNSSLGDLSKIEVLDISWFTECMGAGKPVQVEARHCLVKSCPVIDQYLEPSTVETVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLKTAEKWYRKGFHSLEEVQADNAIHFTKMQKAGFLYYDDISAAVCKAEAQAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKYLKKKTTNNYLALNDVCSDLSEWHYKG

SEQ ID NO: 3 - protein, 510 aa, Chinchilla lanigera
MDPLQAAHSGPRKKRPRQTGTLMVSSPHDVRFGDLVLFILEKKMGTTRRAFLMELARRKGFRVENELSDSVTHIVAENNSGNDVLEWLQVQNIQASSRLELLDVSWLIECMGAGKPVEMTGKHQLLVRRDYPASPKPGPQKTPSLAVQKISEYACQRRTTLNNCNCIFTNAFEILAENCEFRENESSYVTYMRAASVLKSLPFTIISMKDTEGIPCLGEKVKCIIEEIIEDGESSEVNAVLNDERYQSFKLFTSVFGVGLKTSEKWFRMGFRSLNKIKSDKSLKFTRMQKAGFLYYEDLVSCVTRAEAEAVSMLVKEAVWAFLPGAFISMTGGFRRGKEIGHDVDFLITSPEVTEDEKQQLLHKVISLWEKKGLLLYSDLVESTFEKLKLPSRKVDALDHFQKCFLILKLHHQRVDSDKSPQQGGKTWKAIRVDLVVCPYERRAFALLGWTGSRQFERDLRRYATHERKMMLDNHALYDKTKRIFLKAESEEEIFAHLGLDYIEPWERNA

SEQ ID NO: 4 - protein, 511 aa, Otolemur garnettii
MDPLHMAHSGPRKKRPRQTAASMVSTPQDIKFRDLVLFILEKKMGTTRRTFLMELARTKGFRVENEFSDSVTHIIAENNSGSDVLEWIQVQKIKAGSQMEVLDVSWLIECMRAGKPVEMTGKHQLVVRGDYSPSPNPAPQKTPPLAVQKISQYACQRRTTLNNCNHIFTDAFEIMAENYEFRENEGYSAAFMRAASVLKSLPFTIISMKDTEGVPCLGDNVKCIIEEIIEEGESSEVKAVLNDERYQSFKLFTSVFGVGLKTSEKWFRMGFRTLSKIRSDKSLRFTRMQQAGFLYYEDLVSCVTRAEAEAVGVLVKEAVRAFLPDAFVTMTGGFRRGKNIGHDVDFLITSPGSTEEEEQQLLHKIMDLWEKKGLLLYCDLVESTFEKLKLPSRKVDALDHFQKCFLIFKLHHQRVVDSEQSNQQEGKTWKAIRVDLVMCPYERRAYALLGWTGSRQFERDLRRYATHERKMILDNHGLWDKTKRIFLKAESEEEIFAHLGLDYIEPWERNA

SEQ ID NO: 5 - protein, 510 aa, Sus scrofa
MDPPQTVPSSPRKKRPRQVGASMASPAHNIKFRELVLFILEKKMGTTRRTFLMELARRKGFRVENELSDSVTHIVAENNSGSEVLEWLQAQKIRASSQLTLLDVSWLIESMGAGKPVEMTGKHQLVVRTDCSASPSPGSQNTLPPAVKKISQYACQRRTTLNNCNHIFTDAFEVLAENYEFRENETFCLAFMRAASVLKSLPFTIISMKDTEGIPCLGDKVKCVIEEIIEDGESSEVKAVLNDERYQSFKLFTSVFGVGLKTSERWFRMGFRSLSKIRSDKTLKFTRMQKAGFLYYEDLVSCVTRAEAEAVGVLVKEAVQAFLPDAFVTMTGGFRRGKKMGHDVDFLITSPGSTDDEEQQLLPKVVNLWEREGLLLYCDLVESTLEKSKLPSRNVDALDHFQKCFLILKLHHQRVDSGMSSQQEGKTWKAIRVDLVMCPYELRAFALLGWTGSRQFERDLRRYATHERKMILDNHALYDKTKRIFLKAESEEEIFAHLGLDYLEPWERNA

SEQ ID NO: 6 - protein, 509 aa, Bos taurus
MDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 7 - DNA, 12 bp, Artificial Sequence
  Synthetic Oligonucleotide
cgttaacatatt

SEQ ID NO: 8 - protein, 494 aa, Artificial Sequence
  Synthetic Polypeptide
MLHIPIFPPIKKRQKLPESRNSCKYEVKFSEVAIFLVERKMGSSRRKFLTNLARSKGFRIEDVLSDAVTHVVAENNSADELLQWLQNSSLGDLSKIEVLDISWFTECMGAGKPVQVEARHCLVKSCPVIDQYLEPSTVETVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLRTAEKWYRKGFHSLEEVQADNAIHFTKMQKAGFLYYDDISAAVCKAEAQAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIDPWQRNA